(19) United States
(12) Patent Application Publication
(10) Pub. No.: US 2020/0352987 A1
(43) Pub. Date: Nov. 12, 2020

(54) EMULSIONS FOR OPHTHALMIC DELIVERY OF ANTIOXIDANTS

(71) Applicant: Tathagata DUTTA, BERHAMPORE (IN)

(72) Inventor: Tathagata DUTTA, BERHAMPORE (IN)

(21) Appl. No.: 16/641,403

(22) PCT Filed: Mar. 26, 2018
(86) PCT No.: PCT/IN2018/050170

§ 371 Date: Feb. 24, 2020

(30) Foreign Application Priority Data
Aug. 30, 2017 (IN) 201741030659

(51) Int. Cl. A61K 33/04; A61K 9/00; A61K 9/107; A61K 31/047; A61K 31/015; A61K 47/44; A61P 27/02; A61P 39/06

(52) U.S. Cl. CPC A61K 33/04; A61K 9/0048; A61K 9/107; A61P 39/06; A61K 31/015; A61K 47/44; A61P 27/02; A61K 31/047

(57) ABSTRACT

An ophthalmic emulsion for ocular delivery of antioxidants comprising one or more antioxidants, at least one oil of vegetable, mineral or animal origin, at least one surfactant and at least one pharmaceutically acceptable excipient, for effective management of Age Related Macular Degenaration (ARMD) and its subsequent clinical manifestations and complications.

### CROSS-REFERENCE TO RELATED APPLICATIONS

This application is a 371 national stage entry of international Application No. PCT/IN2018/050170 filed on Mar. 26, 2018, the entire content of which is hereby incorporated by reference. This application claims priority to India Patent Application No. 201741030659 filed on Aug. 30, 2017, and to PCT Application No. PCT/IN2018/050170 filed on Mar. 26, 2018, the entire contents of which are hereby incorporated by reference.

### FIELD OF THE INVENTION

This invention relates to drug delivery dosage forms and methods to treat medical conditions of the eye. Specifically, this invention relates to emulsion drug delivery dosage forms of antioxidants for drug delivery within the eye, for effective management of Age Related Macular Degenaration (ARMD) and its subsequent clinical manifestations and complications.

### BACKGROUD

Age related macular degeneration (ARMD) is a severe problem of the eye which accounts for the loss of vision of huge number of elderly population across the globe. The disease which starts as a mild innocent problem of the eye, including occasional floaters and black dots in front of the eye gradually progresses to the loss of peripheral vision to complete loss of vision

Antioxidants are well known to slow down the progression of the ARMD disease. There are several formulations available in the market containing single or combination of antioxidants like Lutein, Zeaxanthin, Beta Carotene, Selenium, etc for oral administration for slowing down the progression of the disease. There are several drawbacks with orally administered drugs or bioactives as the drug does not reach the eye at appropriate concentrations and has either none or very poor pharmacological action in the eye when administered through oral route.

Hence, there is a need for a topical formulation of antioxidants for ocular administration that will release the drug in a sustained manner in the eye which on repeated administration will lead to a steady concentration of antioxidants in the aqueous and vitreous humor thereby increasing the macular pigment optical density (MPOD), which will be helpful for better management of Age Related Macular Degeneration.

### SUMMARY

Accordingly, the invention provides new topical drug delivery system that releases the active agent in a sustained manner to provide the desired therapeutic effects, and methods of making such systems. The topical drug delivery system of the invention as disclosed herein is an emulsion. The emulsion composition as disclosed herein provides release of the active agent in sustained manner and repeated administration of the formulation several times a day will lead to a steady concentration of antioxidants in the aqueous &vitreous humor increasing the macular pigment optical density (MPOD), which will be helpful for better management of Age Related Macular Degeneration.

The emulsion composition as disclosed herein is a stable ophthalmic emulsion comprising one or more antioxidants, at least one oil from vegetable, mineral or animal origin, at least one surfactant and at least one pharmaceutically acceptable excipient. The concentration of the antioxidant in the emulsion composition as disclosed herein is in the range of about 0.0003%-0.3% w/v and the antioxidant is selected from the group comprising of lutein, zeaxanthin, beta carotene, selenium or a combination thereof.

The oil is a vegetable oil or mineral oil or animal oil or a combination there of. The vegetable oil is selected from the group comprising of Castor Oil, Cotton seed Oil, Peanut Oil, Coconut oil, Rice bran oil, Sunflower oil, Sesame oil, Soybean oil, Flax oil, Canola oil, Olive oil, Mustard Oil, Jojoba oil or a combination there of. The animal oil is selected from the group comprising of fish oil, shark liver oil, cod liver Oil or a combination there of. The mineral oil is Liquid Paraffin.

The surfactant is selected from the group comprising of Polyoxyethylated nonionic surfactants like Polysorbate80, Polysorbate 60, Polysorbate, 40, Polysorbate 20, Cremophors, Tyloxapols, Poloxamers, Benzalkonium chloride, Benzethonium chloride, Cetyl alcohol, Carbomer, Cholesterol, Cocamidopropyl betaine, glyceryl tnonostearate, lanolin alcohols, lauralkonium chlorides, N lauroylsarcosine, Nonoxynol 9, Octoxynol 40, Polyoxyl 35 castor oil, Polyoxyl 40 hydrgenated castor oil. Polyoxyl 40 stearate, Sorbitanmonolaureate, Sorbitan monooleate, Sorbitanmonopalmitate, Polyoxyethylene (2) Steryl Ether, Polyoxyethylene (2) Cetyl Ether, Polyoxyethylene (2) Oleyl Ether, Polyeoxyethyllene (2) Nonylphenylether, Polyoxyethylene (2) isooctylphenyl ether, Polyoxyletheylene (4) lauryl ether, Polyoxylethylene (5) isooctylphenylether or a combination thereof.

The emulsion composition as described herein further comprises at least one pH adjusting agent, at least one buffering agent, at least one osmolarity control agent and at least one antimicrobial preservative.

The pH adjusting agent selected from the group comprising of Hydrochloric Acid, Sodium Hydroxide, Sulphuric Acid, Sodium Sulphate, Acetic Acid, Sodium Citrate, Ammonium Hydroxide, Citric Acid, Diethanolamine, Nitric Acid, Phosphoric Acid or a combination thereof.

The buffering agent selected from the group comprising of Acetic Acid, Boric Acid, Citric Acid, Phosphoric Acid, Potassium Acetate, Potassium Phosphate, Potassium Sulphate, Potassium Sorbate, Sodium Acetate, Sodium borate, Sodium Carbomate, Sodium Citrate, Sodium Phosphate, Sorbic Acid, Tromethamine or a combination thereof.

The osmolarity control agent selected from the group comprising of Sodium Chloride, Sodium Sulphate, Sodium Nitrate, Sorbitol, Mannitol, Calcium Chloride, Glycerin, Magnesium Chloride, PEG 300, PEG 400, Potassium Chloride, Propylene Glycol or a combination thereof.

The antimicrobial preservative selected from the group comprising of Quaternary ammonium compounds selected from Benzalkonium Chloride, Benzethonium chloride, Benzododecinium bromide or Polyquatermium-1; or Acid/Base compounds selected from Boric acid, sodium acetate or sodium borate; or Alcohols selected from chlorobutanol or Phenylethyl alcohol; or Organic Mercuric compounds selected from Phenyl mercuric acetate, Phenyl mercuric nitrate or Thimerosal; or Parabens selected from methyl paraben or Propyl Paraben; or Oxidizing agent sodium chlorite; or Metal salt Zinc Chloride or a combination thereof.

### DETAILED DESCRIPTION

As described herein, the disclosure provides emulsion compositions for ocular delivery of antioxidants that releases the active agent in sustained manner and repeated administration of the formulation several times a day will lead to a steady concentration of antioxidants in the aqueous and vitreous humor increasing the macular pigment optical density (MPOD), which will be helpful for better management of Age Related Macular Degeneration.

The emulsion composition as disclosed herein is a stable oil-in-water ophthalmic emulsion comprising one or more antioxidants, at least one oil from vegetable, mineral or animal origin, at least one surfactant and at least one pharmaceutically acceptable excipient

The antioxidant is selected from the group comprising of lutein, zeaxanthin, beta carotene, selenium or a combination there of. The concentration of the antioxidant in the emulsion composition as disclosed herein is in the range of about 0.0003%-0.03% w/v.

The emulsion composition as described herein further comprises at least one pH adjusting agent, at least one buffering agent, at least one osmolarity control agent and at least one antimicrobial preservative.

The oil is a vegetable oil or mineral oil or animal oil or a combination there of. The vegetable oil is selected from the group comprising of Castor Oil, Cotton seed Oil, Peanut Oil, Coconut oil, Rice bran oil, Sunflower oil, Sesame oil, soya been oil, Flax oil, Canola oil, Olive oil, Mustard Oil, Jojoba oil or a combination there of. The animal oil is selected from the group comprising of fish oil, shark liver oil, cod liver oil or a combination there of. The mineral oil is Liquid Paraffin.

The surfactant is selected from the group comprising of Polyoxyethylated nonionic surfactants like Polysorbate80, Polysorbate 60, Polysorbate, 40, Polysorbate 20, Cremophors, Tyloxapols, Poloxamers, Benzalkonium chloride, Benzethonium chloride, Cetyl alcohol, Carbomer, Cholesterol, Cocamidopropyl betaine, glyceryl monostearate, lanolin alcohols, lauralkonium chlorides, N lauroylsarcosine, Nonoxynol 9, Octoxynol 40, Polyoxyl 35 castor oil, Polyoxyl 40 hydrgenated castor oil. Polyoxyl 40 stearate, Sorbitanmonolaureate, Sorbitan monooleate, Sorbitanmonopalmitate, Polyoxyethylene (2) Steryl Ether, Polyoxyethylene (2) Cetyl Ether, Polyoxyethylene (2) Oleyl Ether, Polyeoxyethyllene (2) Nonylphenylether, Polyoxyethylene (2) isooctylphenyl ether, Polyoxyletheylene (4) lauryl ether, Polyoxylethylene (5) isooctylphenylether or a combination thereof.

In one embodiment, the topical ocular suspension comprises the antioxidants lutein and zeaxanthin and pharmaceutical excipients.

### EXAMPLE: 1

Lutein+Zeaxanthin Ophthalmic Emulsion

| S. No | Ingredient | mg/mL | |
|---|---|---|---|
| 1 | Lutein | 0.03 | mg |
| 2 | Zeaxanthin | 0.03 | mg |
| 3 | Seasame Oil | 36 | mg |
| 4 | Polysorbate 80 | 24 | mg |
| 5 | Hydroxypropyl | 10 | mg |
| | Methyl Cellulose (HPMC) | | |
| 6 | Benzalkonium Chloride | 1 | mg |
| 7 | Water for Injection | q.s | |
| 8 | Sodium citrate | 20 | mg |

| | | |
|---|---|---|
| 9 | Hydrochloric acid/sodium hydroxide | q.s |

Process: Seasame oil was heated to about 70° C. and to it was added Lutein & Zeaxanthin and stirred to completely dissolve the same in the oil phase. Sterile purified water was heated to about 70° C., and to it polysorbate 80, Sodium citrate, Benzalkonium Chloride and HPMC was added and mixed to form the aqueous phase. The oil phase was added while stiring the aqueous phase with a high shear mixerto form the emulsion. This crude emulsion was finely divided in a high pressure homogenizer and the volume was made up with sufficient quantity of water for injection and the pH was adjusted using either sodium hydroxide/hydrochloric acid and sterilized by filtration through 0.22 μm membrane filter to form the sterile ophthalmic emulsion of antioxidants, which is filled into containers aseptically and sealed.

In another embodiment, the ophthalmic emulsion comprises the antioxidants lutein, zeaxanthin, alpha tocopherol, and selenium and pharmaceutical excipients.

### EXAMPLE: 2

Lutein, Zeaxanthin, & Selenium Ophthalmic Emulsion

| S. No | Ingredient | mg/mL | |
|---|---|---|---|
| 1 | Lutein | 0.03 | mg |
| 2 | Zeaxanthin | 0.06 | mg |
| 3 | Selenium | 0.01 | mg |
| 4 | Peanut Oil | 81 | mg |
| 5 | Poloxamer 188 | 56 | mg |
| 6 | Polyethylene Glycol (PEG) 400 | 20 | mg |
| 7 | Sodium chloride | 9 | mg |
| 8 | Phenyl mercuric nitrate | 2 | mg |

| | | |
|---|---|---|
| 9 | Water for Injection | q.s |
| 10 | Sodium Hydroxide/Hydrochloric Acid | q.s |

Process: Peanut oil was heated to about 70° C. and to it was added Lutein, Zeaxanthin and stirred to completely dissolve the same in the oil phase. Sterile purified water was heated to about 70° C., and to it Selenium, Poloxamer 188, Polyethylene Glycol (PEG) 400, Sodium Chloride, and Phenyl mercuric nitrate was added and mixed to form the aqueous phase. The oil phase was added while stirring the aqueous phase with a high shear mixer to form the emulsion. This crude emulsion was finely divided in a high pressure homogenizer to form a stable emulsion of antioxidants and the volume was made up with sufficient quantity of water for injection and the pH was adjusted using either sodium hydroxide/hydrochloric acid and sterilized by filtration through 0.22 μm membrane filter to form the sterile ophthalmic emulsion of antioxidants, which is filled into containers aseptically and sealed.

In another embodiment of the invention, the ophthalmic emulsion comprises lutein, and pharmaceutical excipients.

### EXAMPLE: 3

Lutein Ophthalmic Emulsion

| S. No | Ingredient | mg/mL | |
|---|---|---|---|
| 1 | Lutein | 0.2 | mg |
| 2 | Medium Chain Triglycerides (MCT) | 54 | mg |
| 3 | Poloxamer 407 | 39 | mg |
| 4 | Sodium Citrate | 0.2 | mg |
| 5 | Citric Acid | 0.6 | mg |
| 6 | Benzalkonium Chloride | 1 | mg |

| | | |
|---|---|---|
| 7 | Hydrochloric Acid/Sodium Hydroxide | q. s |
| 8 | Water for Injection | q. s |

Process: Medium Chain Triglycerides was heated to about 70° C. and to it was added Lutein, and stirred to completely dissolve the same in the oil phase. Sterile purified water was heated to about 70° C,and to it Poloxamer 407, Sodium Citrate, Citric Acid, and Benzalkonium Chloride was added and mixed to form the aqueous phase. The oil phase was added while stirring the aqueous phase with a high shear mixer to form the emulsion. This crude emulsion was finely divided in a high pressure homogenizer to form a stable emulsion of antioxidants and the volume was made up with sufficient quantity of water for injection and the pH was adjusted using either sodium hydroxide/hydrochloric acid and sterilized by filtration through 0.22 μm membrane filter to form the sterile ophthalmic emulsion of antioxidants, which is filled into containers aseptically and sealed.

In another embodiment of the invention, the ophthalmic emuslion comprises of beta carotene and pharmaceutical excipients.

### EXAMPLE: 4

Beta Carotene Ophthalmic Emulsion

| S. No | Ingredient | mg/mL | |
|---|---|---|---|
| 1 | Beta Carotene | 0.09 | mg |
| 2 | Fish Oil | 60 | mg |
| 3 | Kolliphor EL (Polyethoxylated Castor Oil) | 54 | mg |
| 4 | Sodium Citrate | 0.2 | mg |
| 5 | Citric Acid | 0.6 | mg |
| 6 | Benzalkonium Chloride | 1 | mg |

| | | |
|---|---|---|
| 7 | Hydrochloric Acid/Sodium Hydroxide | q. s |
| 8 | Water for Injection | q. s |

Process: Fish Oil was heated to about 70° C. atto it was added Beta carotene and stirred to completely dissolve the same in the oil phase. Sterile purified water was heated to about 70° C., and to it Kolliphor EL, Sodium Citrate, Citric Acid, and Benzalkonium Chloride were added and mixed to form the aqueous phase. The oil phase was added while stirring the aqueous phase with a high shear mixer to form the emulsion. This crude emulsion was finely divided in a high pressure homogenizer to form a stable emulsion of antioxidants and the volume was made up with sufficient quantity of water for injection and the pH was adjusted using either sodium hydroxide/hydrochloric acid and sterilized by filtration through 0.22 μm membrane filter to form the sterile ophthalmic emulsion of antioxidants, which is filled into containers aseptically and sealed.

In another embodiment of the invention, the ophthalmic emulsion comprises of zeaxanthin and pharmaceutical excipients.

### EXAMPLE: 5

Zeaxanthin Ophthalmic Emulsion

| S. No | Ingredient | mg/mL | |
|---|---|---|---|
| 1 | Zeaxanthin | 0.06 | mg |
| 2 | Liquid Paraffin | 69 | mg |
| 3 | Polyethylene glycol oleyl ether | 54 | mg |
| | (Brij® 93) | | |
| 4 | Sodium borate | 2 | mg |
| 5 | Boric acid | 6 | mg |
| 6 | Benzalkonium Chloride | 1 | mg |
| 7 | Sorbitol | 12 | mg |

| | | |
|---|---|---|
| 8 | Hydrochloric Acid/Sodium Hydroxide | q.s |
| 9 | Water for Injection | q.s |

RF-201741030659

Process: Liquid Paraffin was heated to about 70° C. and to it was added Zeaxanthin and stirred to completely dissolve the same in the oil phase. Sterile purified water was heated to about 70° C., and to it Polyethylene glycol oleyl ether (Brij® 93), Sodium Borate, Boric Acid, Sorbitol and Benzalkonium Chloride was added and mixed to form the aqueous phase. The oil phase was added to the aqueous phase while stirring the aqueous phase with a high shear mixer to form the emulsion. This crude emulsion was finely divided in a high pressure homogenizer to form a stable emulsion of antioxidants and the volume was made up with sufficient quantity of water for injection and the pH was adjusted using either sodium hydroxide/hydrochloric acid and sterilized by filtration through 0.22 μm membrane filter to form the sterile ophthalmic emulsion of antioxidants, which is filled into containers aseptically and sealed.

In another embodiment of the invention, the ophthalmic emulsion comprises of lutein and pharmaceutical excipients.

### EXAMPLE: 6

Lutein Ophthalmic Emulsion

| S. No | Ingredient | mg/mL | |
|---|---|---|---|
| 1 | Lutein | 0.3 | mg |
| 2 | Jojoba oil | 60 | mg |
| 3 | Polyethylene glycol oleyl ether | 45 | mg |
| | (Brij® 93) | | |
| 4 | Sodium borate | 2 | mg |
| 5 | Boric acid | 6 | mg |
| 6 | Benzalkonium Chloride | 1 | mg |
| 7 | Sorbitol | 12 | mg |

| | | |
|---|---|---|
| 8 | Hydrochloric Acid/Sodium Hydroxide | q.s |
| 9 | Water for Injection | q.s |

Process: Liquid Paraffin was heated to about 70° C. and to it was added Lutein and stirred to completely dissolve the same in the oil phase. Sterile purified water was heated to about 70° C., and to it Polyethylene glycol oleyl ether (Brij® 93), Sodium Borate, Boric Acid, Sorbitol and Benzalkonium Chloride were added and mixed to form the aqueous phase. The oil phase was added to the aqueous phase while stirring the aqueous phase with a high shear mixer to form the emulsion. This crude emulsion was finely divided in a high pressure homogenizer to form a stable emulsion of antioxidants and the volume was made up with sufficient quantity of water for injection and the pH was adjusted using either sodium hydroxide/hydrochloric acid and sterilized by filtration through 0.22 μm membrane filter to form the sterile ophthalmic emulsion of antioxidants, which is filled into containers aseptically and sealed.

The method for preparing the topical ocular suspension according to the disclosure described herein is not limited to the above methods. The ophthalmic emulsion for ocular delivery of antioxidants according to the disclosure described herein can be prepared by using various other techniques.

1-12. (canceled)

13. A stable oil-in-water ophthalmic emulsion for ocular delivery of antioxidants comprising one or more antioxidants as an active pharmaceutical ingredient, at least one oil of vegetable, mineral or animal origin or a combination thereof, at least one surfactant and at least one pharmaceutically acceptable excipient, for effective management of Age Related Macular Degenaration (ARMD) and its subsequent clinical manifestations and complications; wherein, the antioxidant is selected from the group comprising of lutein, zeaxanthin, beta carotene, selenium or a combination thereof.

14. The stable oil-in-water ophthalmic emulsion of claim 13, wherein the concentration of antioxidant is in the range of about 0.0003-0.03% w/v.

15. The stable oil-in-water ophthalmic emulsion of claim 13, wherein the oil is a vegetable oil or mineral oil or animal oil or a combination thereof.

16. The stable oil-in-water ophthalmic emulsion of claim 13, wherein the vegetable oil is selected from the group comprising of Castor Oil, Cotton seed Oil, Peanut Oil, Coconut oil, Rice bran oil, Sunflower oil, Sesame oil, soybean oil, Flax oil, Canola oil, Olive oil, Mustard Oil, Jojoba oil or a combination thereof.

17. The stable oil-in-water ophthalmic emulsion of claim 13, wherein the animal oil is selected from the group comprising of fish oil, shark liver oil, cod liver oil or a combination thereof.

18. The stable oil-in-water ophthalmic emulsion of claim 13, wherein the mineral oil is Liquid Paraffin and White Mineral Oil or a combination thereof.

19. The stable oil-in-water ophthalmic emulsion of claim 13, wherein the surfactant is selected from the group comprising of Polyoxyethylated nonionic surfactants like Polysorbate80, Polysorbate 60, Polysorbate, 40, Polysorbate 20, Cremophors, Tyloxapols, Poloxamers, Benzalkonium chloride, Benzethonium chloride, Cetyl alcohol, Carbomer, Cholesterol, Cocamidopropyl betaine, glyceryl monostearate, lanolin alcohols, lauralkonium chlorides, N lauroylsarcosine, Nonoxynol 9, Octoxynol 40, Polyoxyl 35 castor oil, Polyoxyl 40 hydrgenated castor oil. Polyoxyl 40 stearate, Sorbitanmonolaureate, Sorbitan monooleate, Sorbitanmonopalmitate, Polyoxyethylene (2) Steryl Ether, Polyoxyethylene (2) Cetyl Ether, Polyoxyethylene (2) Oleyl Ether, Polyeoxyethyllene (2) Nonylphenylether, Polyoxyethylene (2) isooetylphenyl ether, Polyoxyletheylene (4) lauryl ether, Polyoxylethylene (5) isooctyiphenylether or a combination thereof.

20. The stable oil-in-water ophthalmic emulsion of claim 13, farther comprises at least one pH adjusting agent selected from the group comprising of Hydrochloric Acid, Sodium Hydroxide, Sulphuric Acid, Sodium Sulphate, Acetic Acid, Sodium Citrate, Ammonium Hydroxide, Citric Acid, Diethanolamine, Nitric Acid, Phosphoric Acid or a combination thereof.

21. The stable oil-in-water ophthalmic emulsion of claim 13, further comprises at least one buffering agent selected from the group comprising of Acetic Acid, Boric Acid, Citric Acid, Phosphoric Acid, Potassium Acetate, Potassium Phosphate, Potassium Sulphate, Potassium Sorhate, Sodium Acetate, Sodium borate, Sodium Carbonate, Sodium Citrate, Sodium Phosphate, Sorbic Acid, Tromethamine or a combination thereof.

22. The stable oil-in-water ophthalmic emulsion of claim 13, further comprises at least one osmolarity control agent selected from the group comprising of Sodium Chloride, Sodium Sulphate, Sodium Nitrate, Sorbitol, Mannitol, Calcium Chloride, Glycerine, Magnesium Chloride, PEG 300, PEG 400, Potassium Chloride, Propylene Glycol or a combination thereof.

23. The stable oil-in-water ophthalmic emulsion of claim 11, further comprises at least one antimicrobial preservative selected from the group comprising of Quaternary ammonium compounds selected from Benzalkonium Chloride, Benzethonium chloride, Benzododecinium bromide or Polyquatermium-1; or Acid/Base compounds selected from Boric acid, sodium acetate or sodium borate; or Alcohols selected from chlorobutanol or Phenylethyl alcohol; or Organic Mercuric compounds selected from Phenyl mercuric acetate, Phenyl mercuric nitrate or Thimerosal, or Parabens selected from methyl paraben or Propyl Parahen; or Oxidizing agent sodium chlorite; or Metal salt Zinc Chloride or a combination thereof.
